(19) United States
(12) Patent Application Publication
(10) Pub. No.: US 2014/0046410 A1
(43) Pub. Date: Feb. 13, 2014

(54) SYSTEM AND METHOD FOR AVOIDING HAIRLOSS DURING CHEMOTHERAPY

(71) Applicant: DAVID WYATT, Santa Barbara, CA (US)

(72) Inventor: DAVID WYATT, Santa Barbara, CA (US)

(21) Appl. No.: 13/584,374

(22) Filed: Aug. 13, 2012

(51) Int. Cl. A61F 7/10

(52) U.S. Cl. 607/104

(57) ABSTRACT

A system and method for scalp cooling to avoid hair loss in patients undergoing chemotherapy with a cooling bonnet, a recirculating ice-water system with ice water circulation hoses, a reservoir, a circulating pump and a power supply, and the method having the steps of providing a cooling bonnet with a recirculating ice water system and chilling the cooling bonnet; placing the chilled cooling bonnet on the patient's head at least 30 minutes before undergoing chemotherapy; during chemotherapy; and retaining the chilled cooling bonnet on the patient's head for at least 90 minutes after chemotherapy with the recirculating ice water system powered by a conventional electrical power supply or by a vehicle power supply such as a 12Volt connection like a cigarette lighter as need may be

### FIELD OF THE INVENTION

This invention generally relates to systems and methods to avoid or lessen hair loss in patients undergoing chemotherapy as treatment for cancer.

### BACKGROUND OF THE INVENTION

As is well known, patients diagnosed with cancer frequently undergo chemotherapy with the administration of cytotoxic drugs to attack rapidly growing cancer cells. This chemotherapy also attacks normal rapidly growing cells in the body such as those in the hair roots causing hair loss or alopecia. While hair loss is generally not permanent, the total loss of one's hair can have a devastating effect on the patient.

It has been found that scalp cooling or cryotherapy can slow the loss of hair or, in some instances eliminate hair loss altogether. The theory is that as the skin of the scalp cools, the blood vessels contract (vascoconstriction) causing a reduction in blood supply to the hair follicles. This action diminishes the amount of cytotoxic drug in the blood stream that reaches the hair follicles. The result is that the hair follicles will not be as dramatically affected by the cytotoxic drugs and hair loss will be diminished. Products have been introduced for scalp cooling such as the “Penguin Cold Cap” that is gel-filled and pre cooled before placement on the patient. This type of device has a limited time of use before it must be replaced with another chilled cap. Because of the requirement for a separate chilling function and the limited time of use, this device requires the patient to remain at the facility administering chemotherapy much longer than necessary. It also requires a nurse or a family member to be available to remove the warm “Penguin Cold Cap”, secure a new one, and place it on the patient. This must be done every 30 minutes or so.

Chemotherapeutic agents are circulating in the bloodstream from the onset of the drug administration. They remain in the bloodstream for a little less than 90 minutes after chemotherapy has been discontinued. In order to fully protect the hair follicles the cooling process must be started 30 minutes prior to the beginning of the chemotherapy, throughout the entire session of chemotherapy, and for 90 minutes afterwards.

In a typical three-hour chemotherapy session, a total of 10 “Penguin Cold Caps” must be used. This number is predicated on the fact that the cold caps only remain cold for about 30 minutes. This requires that the location administering the chemotherapy have freezer capacity for 10 of these bulky caps, and in addition a nurse or family member that can make these 30 minute changes.

Another product is the DigniCap™ scalp cooling system. This consists of a cooling unit with connected cooling cap. This system is relatively expensive and bulky, limiting its use to medical facilities and suffers from the same disadvantages as the “Penguin Cold Cap” in that the patient must remain at the facility administering chemotherapy longer than necessary.

All of the present systems lack mobility and ease of use, thereby severely limiting the availability and affordability of cryotherapy. These disadvantages are eliminated in the present invention.

### SUMMARY OF THE INVENTION

It is an object of this invention to provide a system and method for scalp cooling to avoid hair loss in patients undergoing chemotherapy, said system and method being convenient, relatively inexpensive and mobile.

It is an object of this invention to provide a self contained cooling system of such size and weight that can be carried by a patient with the cooling system circulating a liquid coolant to a cooling bonnet configured to fit over a human head and scalp. It is also an object of this invention that the self contained cooling system be powered by a conventional electrical power supply or by a vehicle power supply such as a 12Volt connection like a cigarette lighter.

It is a further object of this invention to provide a method for scalp cooling to avoid hair loss in patients undergoing chemotherapy comprising the steps of providing a cooling bonnet with a recirculating ice water system and chilling the cooling bonnet; placing the chilled cooling bonnet on the patient's head at least 30 minutes before undergoing chemotherapy; during chemotherapy; and retaining the chilled cooling bonnet on the patient's head for at least 90 minutes after chemotherapy with the recirculating ice water system powered by a conventional electrical power supply or by a vehicle power supply such as a 12Volt connection like a cigarette lighter as need may be.

### BRIEF DESCRIPTION OF THE DRAWINGS

FIG. 1 shows the cooling bonnet attached to an ice reservoir of the recirculating ice water system by special tubing.

FIG. 2 shows the proper application of the cooling bonnet to the head viewed from the rear.

FIG. 3 shows the proper application of the cooling bonnet to the head viewed from the side.

FIG. 4 shows the proper application of the cooling bonnet to the head viewed from the front.

FIG. 5 shows the head from front with the cooling bonnet closed.

FIG. 6 shows the head from the top with the cooling bonnet closed.

### DETAILED DESCRIPTION OF THE INVENTION

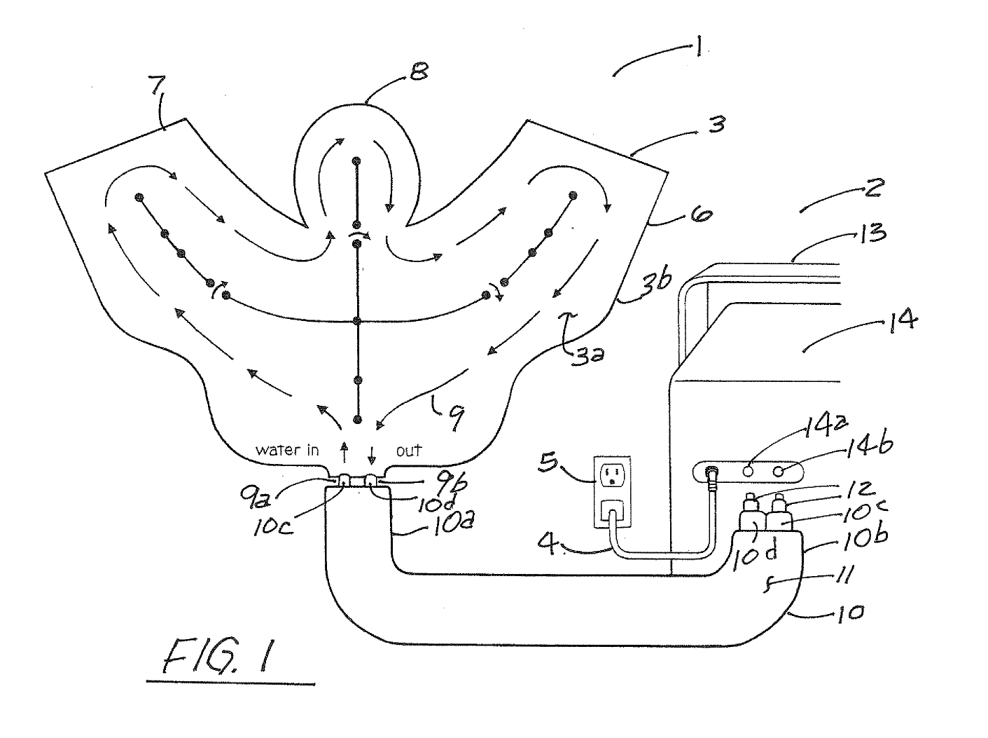

FIG. 1 depicts the inventive scalp cooling system 1 showing the cooling bonnet 3 attached to an ice water circulation hose bundle 10 as part of an ice water recirculating system 2 with an ice reservoir 14 containing a circulating pump not shown and a carrying handle 13. The ice water recirculating system 2 has a power supply cord 4 connected to a power outlet 5. It is intended that the power supply to the ice water recirculating system 2 be adaptable to both conventional A/C electrical power and a low voltage source such as a vehicle 12Volt system that would allow the recirculating ice-water system 2 to function in a vehicle while a patient went home after chemotherapy. The ice water in the reservoir 14 circulates through the hose bundle 10 into the cooling bonnet 3 and back to the reservoir 14 to be re-cooled. The system can cool the scalp for up to 7 hours unattended. It can continue to cool for much longer if the ice of the reservoir is refreshed.

As shown in FIG. 1, the cooling bonnet 3 has a right flap 6, a left flap 7, a middle flap 8, an outer surface 3a, an inner surface 3b, and ice water circulation channel 9 between the outer surface 3a, and inner surface 3b. The ice water circulation channel 9 has an inlet socket 9a and an outlet socket 9b. The ice water circulation hose bundle 10 has a bonnet end 10a and a reservoir end 10b. The ice water circulation hose bundle 10 combines an inlet hose 10c and an outlet hose 10d covered in a suitable insulation 11. At the bonnet end 10a, the inlet hose 10c and an outlet hose 10d each has a snap end 12 for respective insertion into the inlet socket 9a and outlet socket 9b of the ice water circulation channel 9 of the cooling bonnet 3. At the reservoir end 10b the inlet hose 10c and an outlet hose 10d each has a snap end 12 for respective insertion into the inlet socket 14a and outlet socket 14b of the reservoir 14.

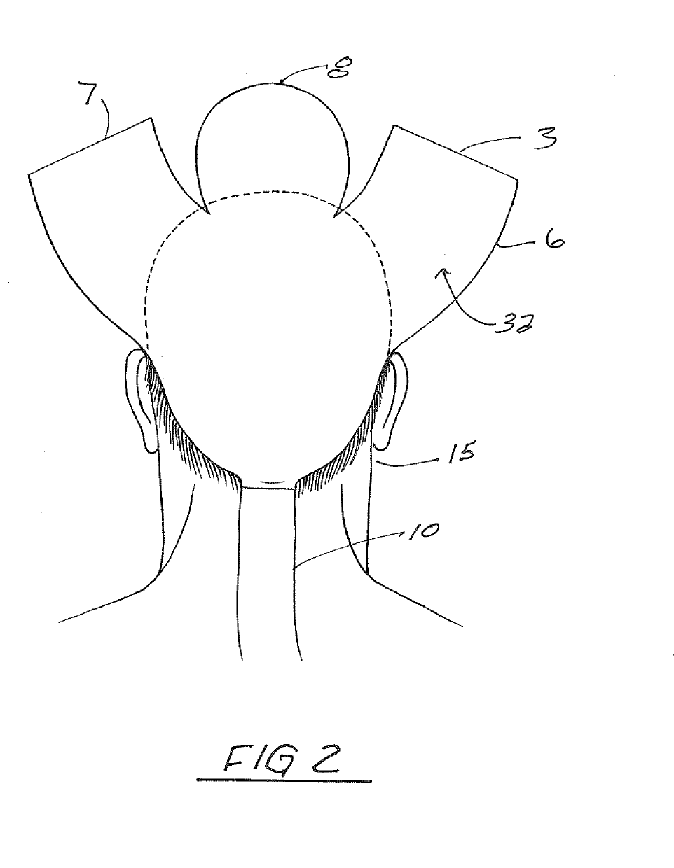

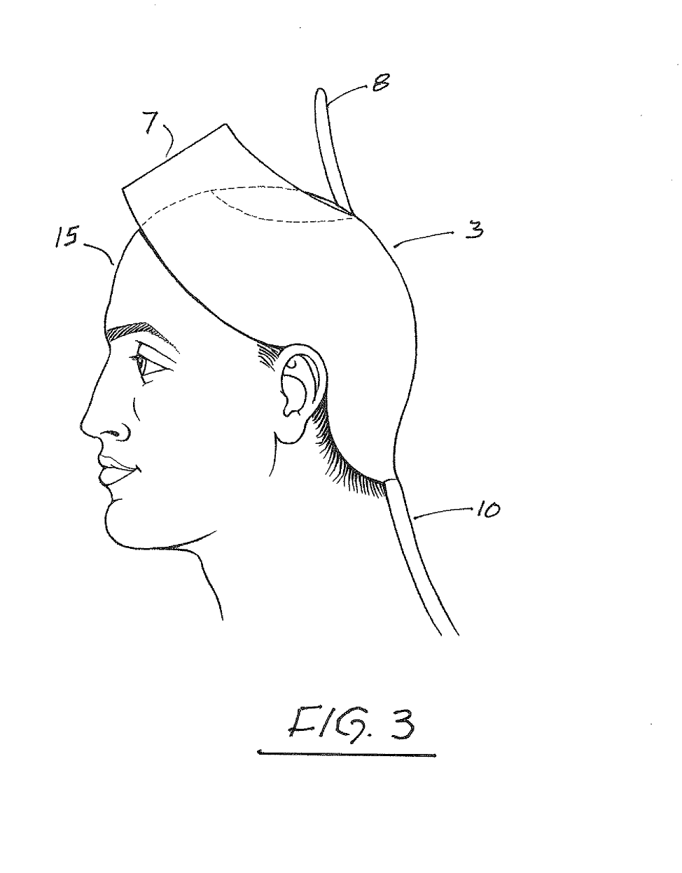

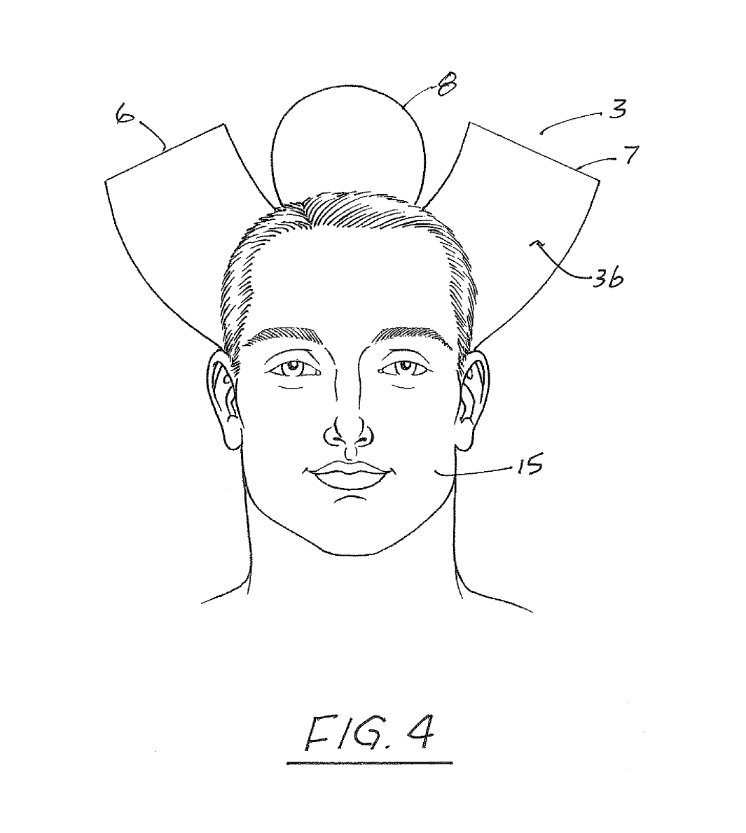

FIGS. 2, 3 and 4 show the proper application of the cooling bonnet 3 to the head 15 of a patient viewed from the rear, side and front of the head 15.

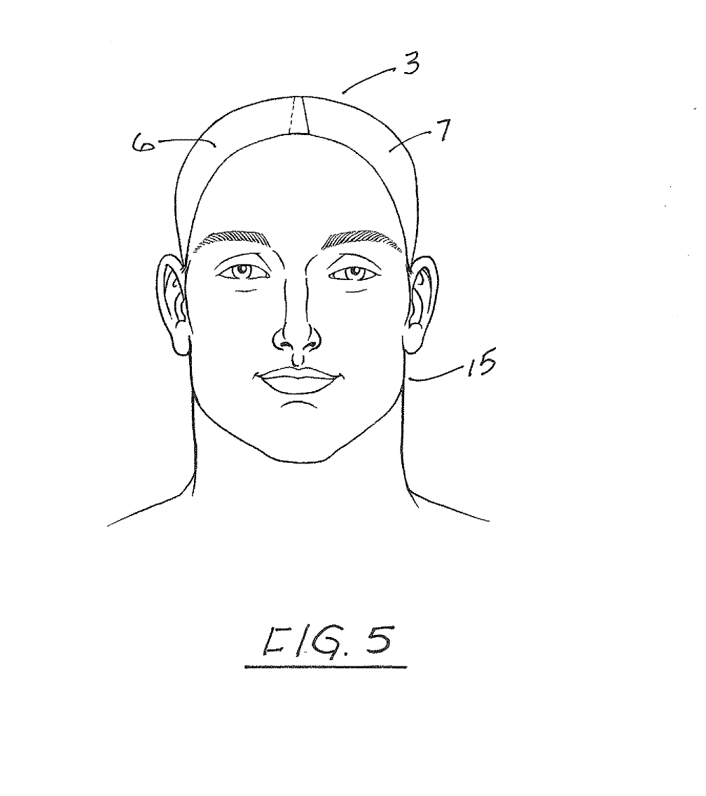

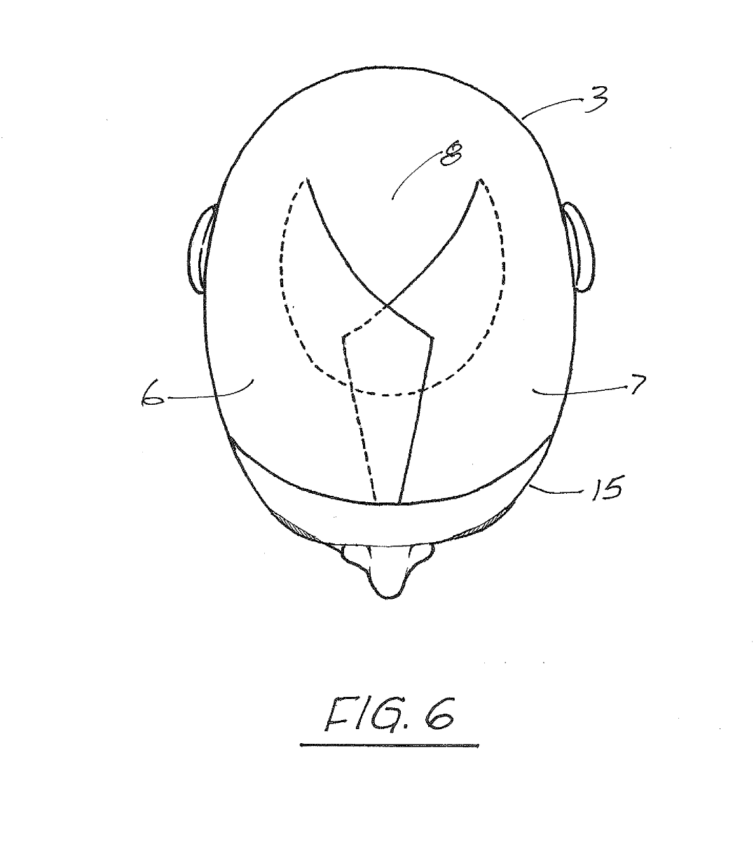

FIGS. 5 and 6 show the cooling bonnet 3 closed on the head 15 as seen from the top and front. As shown, the right flap 6 and the left flap 7 fold over the middle flap 8.

Preferably, and as intended in the inventive system and method to effectively reduce or eliminate hair loss during chemotherapy, the scalp of the patient must be pre-cooled for at least 30 minutes prior to chemotherapy, cooled during chemotherapy and for at least 90 minutes after chemotherapy. By providing a recirculating ice-water system 2 that can be carried by the patient, the patient has the option of leaving the chemotherapy center and returning home to more comfortable surroundings without compromising the benefits of cryotherapy.

1. A system for scalp cooling to avoid hair loss on a patient's head when undergoing chemotherapy, comprising: a cooling bonnet for the patient's head with a right flap, a left flap, a middle flap, an upper surface and a lower surface and an ice water circulation channel between the upper surface and the lower surface, a recirculating ice water system with an ice reservoir, an electric power supply, a circulating pump and ice water circulation hoses to circulate ice water to the ice water circulation channel of the cooling bonnet.

2. The system of claim 1 where the electric power supply is adaptable to both conventional AIC electrical power and a low voltage source.

3. A method for scalp cooling to avoid hair loss on a patient's head when undergoing chemotherapy comprising the following steps:
providing a cooling bonnet for the patient's head with a right flap, a left flap, a middle flap, an upper surface and a lower surface and an ice water circulation channel between the upper surface and the lower surface, a recirculating ice water system with an ice reservoir, an electric power supply, a circulating pump and ice water circulation hoses to circulate ice water to the ice water circulation channel of the cooling bonnet;
cool the scalp of the patient with the cooling bonnet for at least 30 minutes prior to chemotherapy;
cool the scalp of the patient with the cooling bonnet during chemotherapy; and cool the scalp of the patient with the cooling bonnet for at least 90 minutes after chemotherapy.
